(19) United States
(12) Patent Application Publication
(10) Pub. No.: US 2017/0360410 A1
(43) Pub. Date: Dec. 21, 2017

(54) ULTRASOUND DIAGNOSTIC IMAGING APPARATUS

(71) Applicant: Konica Minolta, Inc., Tokyo (JP)

(72) Inventor: Kazushi OTA, Tokyo (JP)

(21) Appl. No.: 15/617,631

(22) Filed: Jun. 8, 2017

(30) Foreign Application Priority Data
Jun. 17, 2016 (JP) 2016-120542

(51) Int. Cl. A61B 8/08; G09G 5/393; A61B 8/14; A61B 8/00

(52) U.S. Cl. CPC A61B 8/5207; A61B 8/467; G09G 5/393; A61B 8/14; A61B 8/465; A61B 8/463; G09G 2360/12

(57) ABSTRACT

An ultrasound diagnostic imaging apparatus which generates ultrasound image data based on a reception signal generated by an ultrasound probe, which transmits and receives ultrasound to and from a test subject, and displays an ultrasound image, includes: a cine-memory that stores cine-image data of the ultrasound image; a determiner that determines whether or not to retain the cine-image data, which is stored in the cine-memory before a transition from a display screen having the ultrasound image into a display screen having a predetermined number of more images; and a hardware processor that indicates an arrangement of the ultrasound image in a display screen transitioned next and a setting state of retention of the cine-image data in the cine-memory before and after the transition, generates a display screen including a first button for accepting an input of the transition into the display screen, and displays the display screen on a display.

The entire disclosure of Japanese Patent Application No. 2016-120542 filed on Jun. 17, 2016 including description, claims, drawings, and abstract are incorporated herein by reference in its entirety.

### BACKGROUND OF THE INVENTION

### Field of the Invention

The present invention relates to an ultrasound diagnostic imaging apparatus.

### Description of the Related Art

In ultrasound diagnosis, heart beats and fetal movements are obtained as ultrasound images with high safety by simply putting an ultrasound probe on a body surface. Thus, examinations can be performed repeatedly. An ultrasound diagnostic imaging apparatus, which is used for performing ultrasound diagnosis and generates and displays an ultrasound image, has been known.

In the field of orthopedic ultrasound, many user scenes exist where two images (two screens), a target part (affected part side) with pain (chief complaint) and the opposite part thereof (healthy part side) in a pair of parts such as hands, are displayed and compared. Whether to acquire an image from of the affected part side or to acquire an image from the healthy part side is case-by-case depending on the condition and state of a patient. In either case, the healthy part side and the affected part side are ultimately in a freeze state of fixed layout in which the healthy part side is on the left/upper side and the affected part side is on the right/lower side or vice versa. The left side part of the body is not displayed in the left image, and the order of arrangement of the healthy part side and the affected part side is preset.

Thus, flexible two-image operation is very important in terms of productivity improvement, and it is necessary to provide highly visible buttons related to the two-image operation so that a user can operate smoothly without confusion.

Moreover, an ultrasound diagnostic imaging apparatus, in which images and buttons are arranged so that the arrangement of the buttons for active (selecting state) setting in a two-image screen is intuitively easily understood, has been known (see JP 2014-147543 A). This apparatus aligns the buttons horizontally when the two images are horizontal and aligns the buttons vertically when the two images are vertical.

In the above conventional ultrasound diagnostic imaging apparatus, the arrangement of the buttons and the screen layout after a transition match, and an active image after two-image display can be thus selected by intuitive operation. However, there has been a problem that a screen layout and which of the images becomes active are unclear upon a transition from a display screen with one ultrasound image into a display screen with two ultrasound images. Moreover, there has been a problem in productivity since a user cannot select which of the affected part side and the healthy part side to start scanning (cine-image data storage) and unnecessary steps increase when the screen layout upon pressing down the two-image buttons does not match the sense of the user.

Therefore, the flexibility of the two-image operation is very important in terms of productivity improvement, and it is necessary to provide highly visible buttons related to the two-image operation so that the user can operate smoothly without confusion.

### SUMMARY OF THE INVENTION

An object of the present invention is to easily designate an active ultrasound image in a display screen of a transition destination and retention of cine-image data before and after the transition upon a transition from a display screen having an ultrasound image into a display screen having more images.

To achieve the abovementioned object, according to an aspect, there is provided an ultrasound diagnostic imaging apparatus which generates ultrasound image data based on a reception signal generated by an ultrasound probe, which transmits and receives ultrasound to and from a test subject, and displays an ultrasound image, and the apparatus reflecting one aspect of the present invention comprises:
a cine-memory that stores cine-image data of the ultrasound image which is live;
a determiner that determines whether or not to retain the cine-image data, which is stored in the cine-memory before a transition from a display screen having the ultrasound image into a display screen having a predetermined number of more images, after the transition; and
a hardware processor that indicates, according to a determination result of the determiner, an arrangement of the ultrasound image, which is active, in a display screen transitioned next and a setting state of retention of the cine-image data in the cine-memory before and after the transition, generates a display screen including a first button for accepting an input of the transition into the display screen, and displays the display screen on a display.

According to an invention of Item. 2, in the ultrasound diagnostic imaging apparatus of Item. 1,
the hardware processor preferably generates a display screen including a second button for accepting an input as to whether or not to retain the cine-image data, which is stored in the cine-memory before the transition, after transition and displays the display screen on the display, and
the determiner preferably determines, according to an input state of the second button, a setting as to whether or not to retain the cine-image data, which is stored in the cine-memory before the transition, after the transition.

According to an invention of Item. 3, in the ultrasound diagnostic imaging apparatus of Item. 2,
the hardware processor preferably sets an input on the second button to be disabled after the transition into the display screen having the predetermined number of the images.

According to an invention of Item. 4, in the ultrasound diagnostic imaging apparatus of any one of Items. 1 to 3,
the hardware processor preferably sets an input on the first button to be disabled for a transition into a display screen, where a same active image is arranged, after the transition into the display screen having the predetermined number of the images.

According to an invention of Item. 5, in the ultrasound diagnostic imaging apparatus of any one of Items. 1 to 4,
the hardware processor preferably divides the cine-memory into a predetermined number of memory regions including a memory region storing last cine-image data before the transition when the cine-image data stored in the cine-memory before the transition is determined to be retained after the transition by the determiner.

According to an invention of Item. 6, in the ultrasound diagnostic imaging apparatus of any one of Items. 1 to 5,
the predetermined number is preferably two.

According to an invention of Item. 7, in the ultrasound diagnostic imaging apparatus of Item. 6,
the hardware processor preferably sets the first button as a button which accepts a transition into a display screen including the ultrasound image and a blank image and accepts an input as to whether to arrange the ultrasound image on a left or right side or on an upper or lower side when the cine-image data stored in the cine-memory before the transition is determined not to be retained after the transition by the determiner, and sets the first button as a button which accepts a transition into a display screen including two ultrasound images, an active ultrasound image and an inactive ultrasound image, and accepts an input as to whether to arrange the active ultrasound image on the left or right side or on the upper or lower side when the cine-image data stored in the cine-memory before the transition is determined to be retained after the transition by the determiner.

According to an invention of Item. 8, in the ultrasound diagnostic imaging apparatus of Item. 6 or 7,
the hardware processor preferably makes the display screen including the first button include a third button which accepts an input as to switch a display between the first button corresponding to the transition into the display screen in which two images are arranged horizontally and the first button corresponding to the transition into the display screen in which the two images are arranged vertically.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is an external view of an ultrasound diagnostic imaging apparatus according to an embodiment of the present invention;

FIG. 2 is a block diagram showing a functional configuration of the ultrasound diagnostic imaging apparatus;

FIG. 3 is a view showing a screen transition between a first display screen and a second display screen;

FIG. 4 is a view showing a screen transition between a third display screen and a fourth display screen;

FIG. 5 is a view showing a screen transition between the first display screen and the third display screen;

FIG. 6 is a view showing a screen transition between the second display screen and the fourth display screen;

FIG. 7 is a view showing a screen transition between the first display screen and a fifth display screen;

FIG. 8 is a view showing a screen transition between the first display screen and a sixth display screen;

FIG. 9 is a view showing a screen transition between the second display screen and a seventh display screen;

FIG. 10 is a view showing a screen transition between the second display screen and an eighth display screen;

FIG. 11 is a view showing a screen transition between the third display screen and a ninth display screen;

FIG. 12 is a view showing a screen transition between the third display screen and a tenth display screen;

FIG. 13 is a view showing a screen transition between the fourth display screen and an eleventh display screen;

FIG. 14 is a view showing a screen transition between the fourth display screen and a twelfth display screen;

FIG. 15 is a view showing a screen transition between the fifth display screen and the tenth display screen;

FIG. 16 is a view showing a screen transition between the sixth display screen and the ninth display screen;

FIG. 17 is a view showing a screen transition between the seventh display screen and the twelfth display screen;

FIG. 18 is a view showing a screen transition between the eighth display screen and the eleventh display screen;

FIG. 19 is a view showing a screen transition between the ninth display screen and the tenth display screen;

FIG. 20 is a view showing a screen transition between the eleventh display screen and the twelfth display screen;

FIG. 21 is a view showing a screen transition between the fifth display screen and the seventh display screen;

FIG. 22 is a view showing a screen transition between the sixth display screen and the eighth display screen;

FIG. 23 is a view showing a screen transition between the ninth display screen and the eleventh display screen;

FIG. 24 is a view showing a screen transition between the tenth display screen and the twelfth display screen;

FIG. 25 is a flowchart showing a horizontal two-image display process; and

FIG. 26 is a flowchart showing a vertical two-image display process.

### DESCRIPTION OF THE PREFERRED EMBODIMENTS

Hereinafter, an embodiment of the present invention will be described in detail with reference to the drawings. However, the scope of the invention is not limited to the illustrated examples.

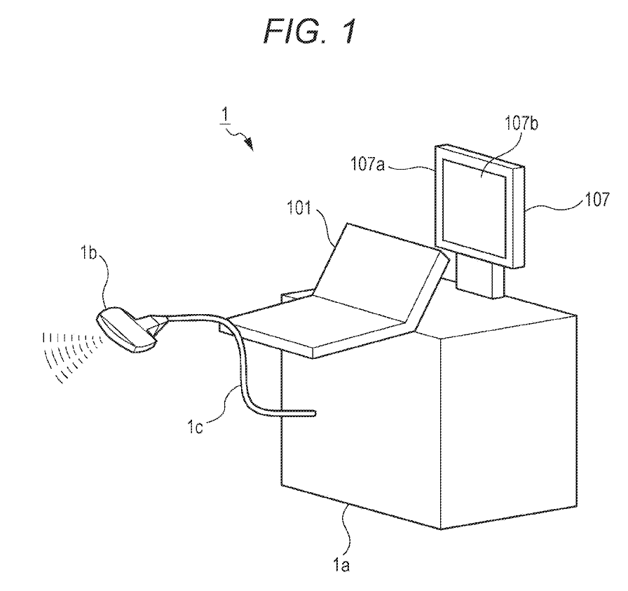

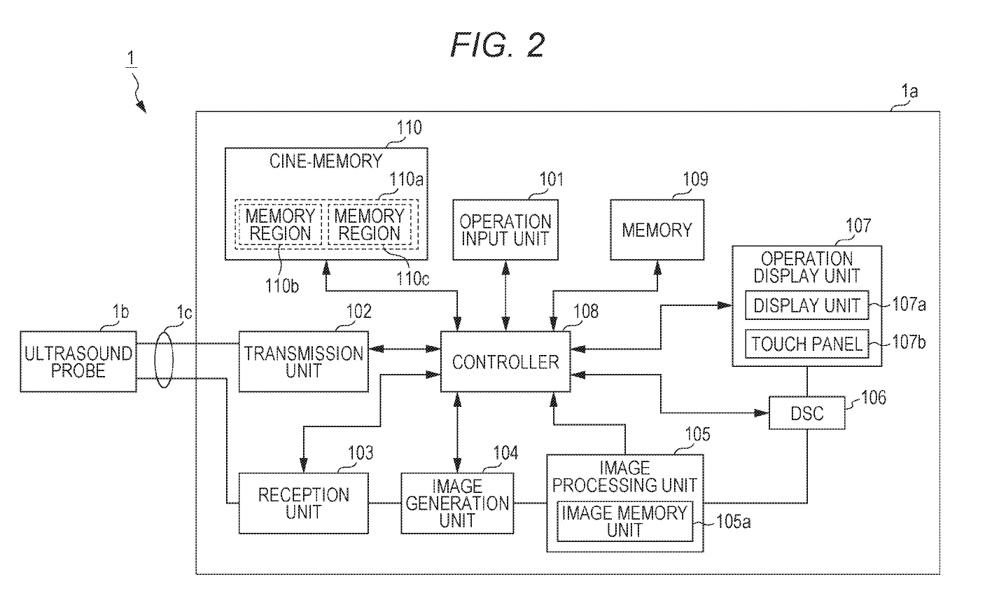

First, a configuration of an apparatus according to the present embodiment will be described with reference to FIGS. 1 and 2. FIG. 1 is an external view of an ultrasound diagnostic imaging apparatus 1 according to the embodiment. FIG. 2 is a block diagram showing a functional configuration of the ultrasound diagnostic imaging apparatus 1.

As shown in FIGS. 1 and 2, the ultrasound diagnostic imaging apparatus 1 of the present embodiment includes an ultrasound diagnostic imaging apparatus main body 1a and an ultrasound probe 1b. The ultrasound probe 1b transmits ultrasound (transmission ultrasound) to a test subject such as an unillustrated living body as well as receives a reflected wave (reflected ultrasound: echo) of the ultrasound reflected on this test subject. The ultrasound diagnostic imaging apparatus main body 1a is connected to the ultrasound probe 1b through a cable 1c and transmits a driving signal, an electric signal, to the ultrasound probe 1b so that the ultrasound probe 1b transmits the transmission ultrasound to the test subject as well as images an inner state of the test subject as an ultrasound image based on a reception signal, an electric signal, generated by the ultrasound probe 1b according to the reflected ultrasound from the test subject received by the ultrasound probe 1b.

The ultrasound probe 1b includes an oscillator constituted by piezoelectric elements, and this oscillator is, for example, aligned plurally in a one-dimensional array in an orientation direction. In the present embodiment, for example, the ultrasound probe 1b including 192 oscillators is used. Note that the oscillators maybe aligned in a two-dimensional array. Moreover, the number of oscillators can be set arbitrarily. Furthermore, in the present embodiment, a linear scanning type electronic scanning probe is employed as the ultrasound probe 1b. However, any one of an electronic scanning type or a mechanical scanning type can be employed, and any type among a linear scanning type, a sector scanning type or a convex scanning type can also be employed.

As shown in FIG. 2, the ultrasound diagnostic imaging apparatus main body 1a includes, for example, an operation input unit 101, a transmission unit 102, a reception unit 103, an image generation unit 104, an image processing unit 105, a digital scan converter (DSC) 106, an operation display unit 107, a controller 108 serving as a determiner and a control unit, a memory 109, and a cine-memory 110 serving as a cine-storage unit.

The operation input unit 101 includes, for example, various switches, buttons, a trackball, a mouse, a keyboard and the like to input a command to instruct a start of diagnosis, data of personal information of the test subject, and the like, and outputs an operation signal to the controller 108.

The transmission unit 102 is a circuit which supplies the driving signal, an electric signal, to the ultrasound probe 1b through the cable 1c according to control by the controller 108 so that the ultrasound probe 1bgenerates the transmission ultrasound. Moreover, the transmission unit 102 includes, for example, a clock generation circuit, a delay circuit and a pulse generation circuit. The clock generation circuit is a circuit which generates a clock signal to determine the transmission timing and transmission frequency of the driving signal. The delay circuit is a circuit which sets a delay time for the transmission timing of the driving signal for each individual path corresponding to each oscillator and delays the transmission of the driving signal by the set delay time to focus the transmission beams constituted by the transmission ultrasound. The pulse generation circuit is a circuit which generates a pulse signal as the driving signal in a predetermined cycle. For example, the transmission unit 102 configured as described above drives some of the consecutive oscillators (e.g., 64 oscillators) among the plurality of the oscillators (e.g., 192 oscillators) aligned in the ultrasound probe 1b to generate the transmission ultrasound. Then, each time the transmission ultrasound is generated, the transmission unit 102 shifts the driving oscillators in the orientation direction to scan.

The reception unit 103 is a circuit which receives the reception signal, an electric signal, from the ultrasound probe 1b through the cable 1c according to control by the controller 108. The reception unit 103 includes, for example, an amplifier, an A/D conversion circuit and a phase adding circuit. The amplifier is a circuit which amplifies the reception signal by a preset amplification factor for each individual path corresponding to each oscillator. The A/D conversion circuit is a circuit which performs A/D conversion on the amplified reception signal. The phase adding circuit is a circuit which provides a delay time for the A/D converted reception signal for each individual path corresponding to each oscillator to adjust the time phases, and adds these up (phase addition) to generate sound ray data.

The image generation unit 104 performs envelope detection processing and log compression on the sound ray data from the reception unit 103 and adjusts the dynamic range and gain to convert the luminance, thereby generating B-mode image data. In other words, the B-mode image data shows the intensity of the reception signal by the luminance. The image generation unit 104 may generate A-mode image data, M-mode image data and image data by the Doppler method, in addition to the B-mode image data.

The image processing unit 105 includes an image memory unit 105a configured with a semiconductor memory such as a dynamic random access memory (DRAM). The image processing unit 105 stores the B-mode image data outputted from the image generation unit 104 in the unit of frames in the image memory unit 105a. The image data in the unit of frames may be called ultrasound image data or frame image data. The frame image data stored in the image memory unit 105a is transmitted to the DSC 106 according to control by the controller 108.

The DSC 106 performs coordinate transformation and the like on the frame image data received from the image processing unit 105 to convert into an image signal for a display unit 107a and outputs the signal to the operation display unit 107.

The operation display unit 107 includes the display unit 107a and a touch panel 107b. The display unit 107a can be a display apparatus such as a liquid crystal display (LCD), a cathode-ray tube (CRT) display, an organic electronic luminescence (EL) display, an inorganic EL display or a plasma display. The display unit 107a displays an image on a display screen according to the image signal outputted from the DSC 106. The touch panel 107b is a pressure sensitive (resistive film pressure) touch panel, in which transparent electrodes are arranged in grid, configured on the display screen of the display unit 107a. The touch panel 107b detects the XY coordinate of the point pressed with a finger on the screen by the voltage value and outputs the detected position signal as the operation signal to the controller 108. Note that the touch panel is not limited to a pressure sensitive type and a type may be selected from various types such as an electrostatic capacitance type for use as appropriate.

The controller 108 is configured by including, for example, a central processing unit (CPU), a read only memory (ROM) and a random access memory (RAM). The controller 108 reads out various processing programs such as a system program stored in the ROM to expand in the RAM and centrally controls the operation of each unit of the ultrasound diagnostic imaging apparatus 1 according to the expanded program. The ROM configured with a nonvolatile memory such as a semiconductor and the like and stores, for example, a system program compatible with the ultrasound diagnostic imaging apparatus 1, various processing programs which can be executed on the system program and execute processings such as image file generation processing, plural image control processing and the like, which will be described later, various data such as a gamma table, and the like. These programs are stored in a format of a program code readable by a computer, and the CPU sequentially executes operations according to the program code. The RAM forms a work area which temporarily stores various programs executed by the CPU and the data related to these programs.

The memory 109 is configured with, for example, a large capacity recording medium such as a hard disk drive (HDD) and stores ultrasound image data saved in association with patient information, and the like.

The cine-memory 110 is, for example, a first in first out (FIFO) memory which is configured with a RAM and the like and stores image data of a live moving image updated in real time as cine-image data according to control by the controller 108. The cine-memory 110 has a memory region 110a for, for example, up to 500 frames of the cine-image data. Moreover, as the memory region of the cine-memory 110, the memory region 110a can also be divided into two regions, a memory region 110b and a memory region 110c, which independently store, for example, up to 250 frames of the cine-image data. Note that the numbers of the frames in the memory regions 110a, 110b and 110c are not limited to 500 and 250 each and may be different numbers of frames as long as the memory capacity allows. Furthermore, the cine-memory 110 may be configured with a part of the RAM of the controller 108.

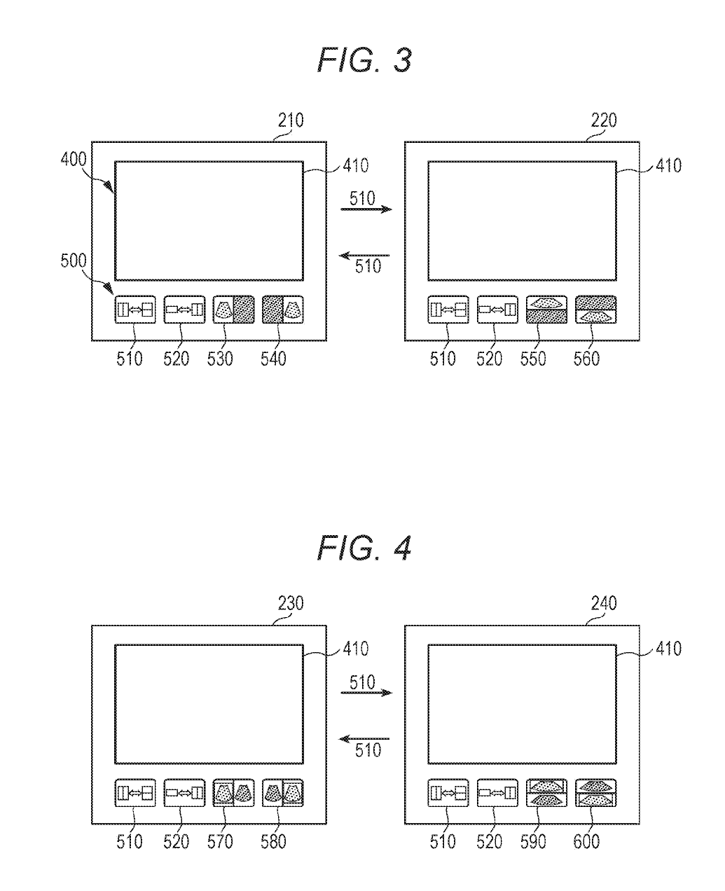

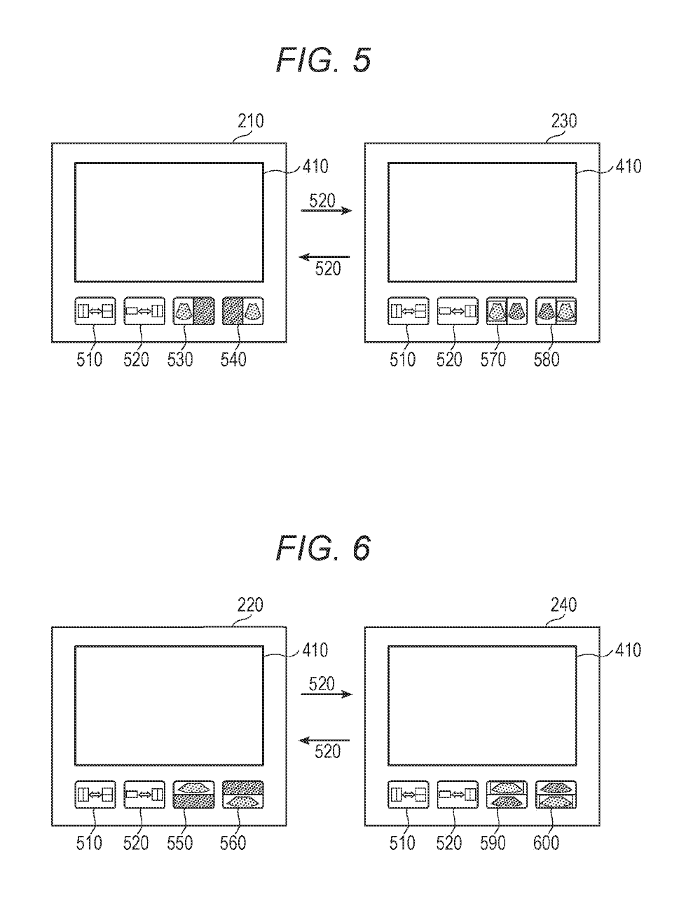

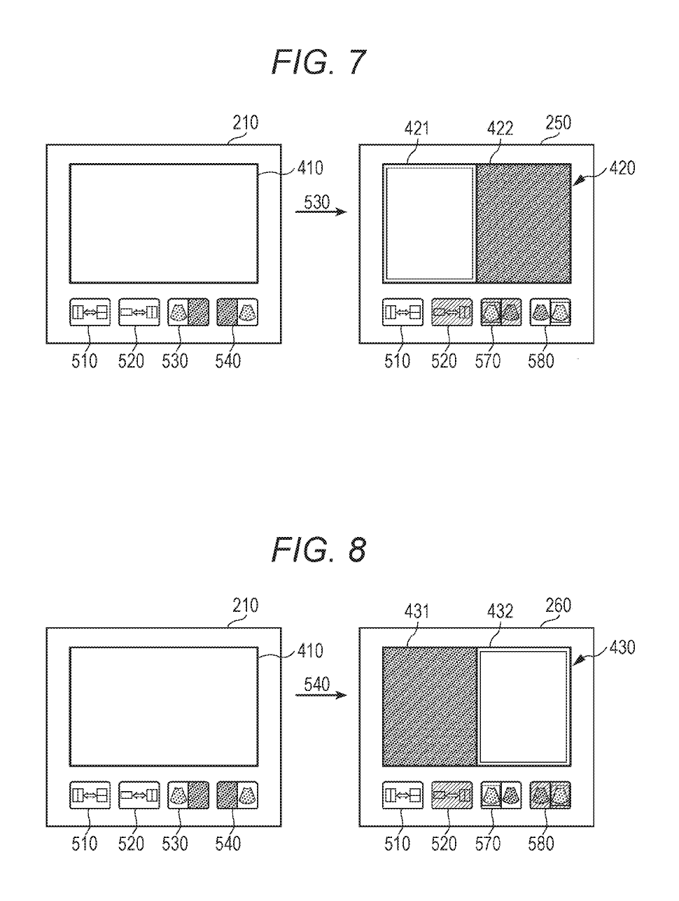

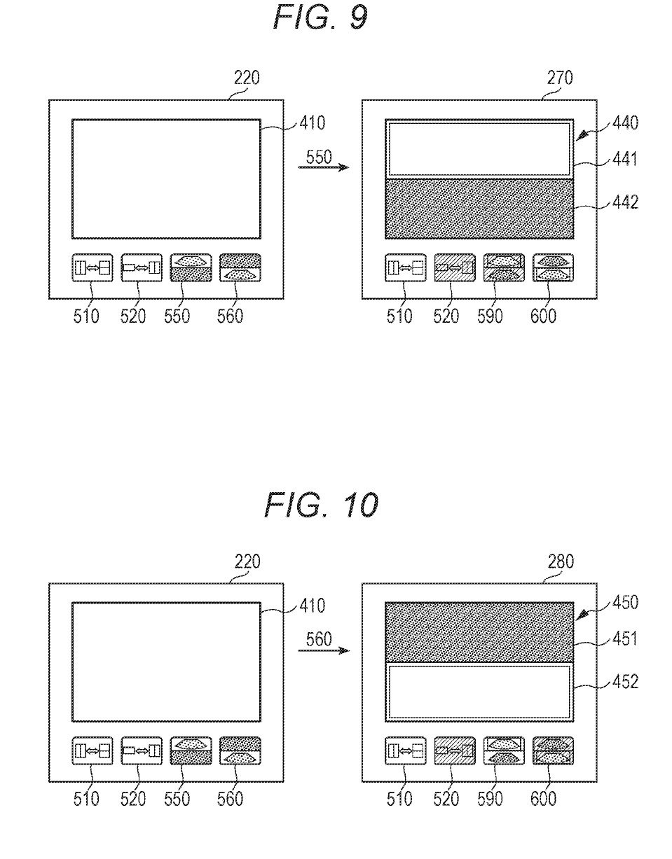

Next, the operations of the ultrasound diagnostic imaging apparatus 1 will be described with reference to FIGS. 3 to 26. First, display screens displayed on the display unit 107a of the ultrasound diagnostic imaging apparatus 1 and screen transitions will be described with reference to FIGS. 3 to 24. FIG. 3 is a view showing a screen transition between a display screen 210 and a display screen 220. FIG. 4 is a view showing a screen transition between a display screen 230 and a display screen 240. FIG. 5 is a view showing a screen transition between the display screen 210 and the display screen 230. FIG. 6 is a view showing a screen transition between the display screen 220 and the display screen 240. FIG. 7 is a view showing a screen transition between the display screen 210 and a display screen 250. FIG. 8 is a view showing a screen transition between the display screen 210 and a display screen 260. FIG. 9 is a view showing a screen transition between the display screen 220 and a display screen 270. FIG. 10 is a view showing a screen transition between the display screen 220 and a display screen 280.

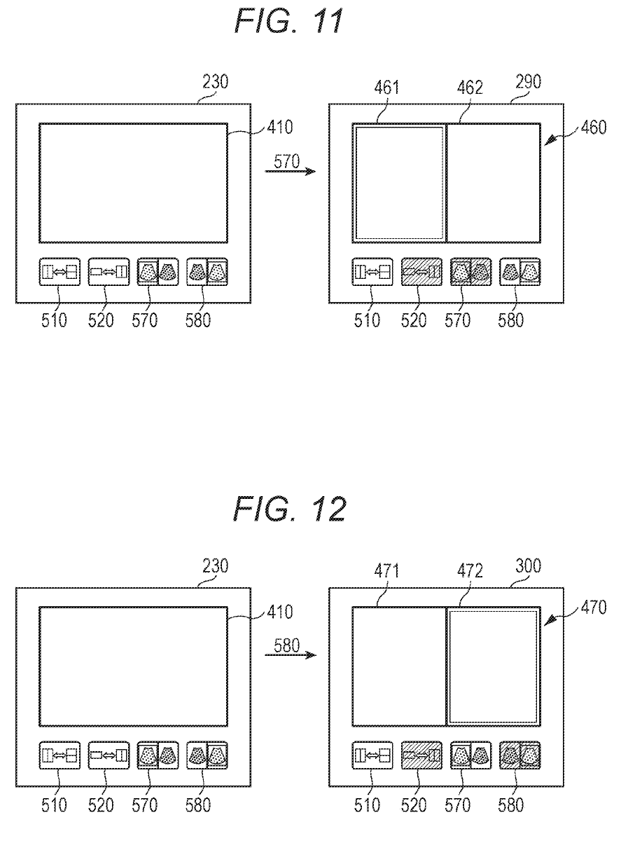

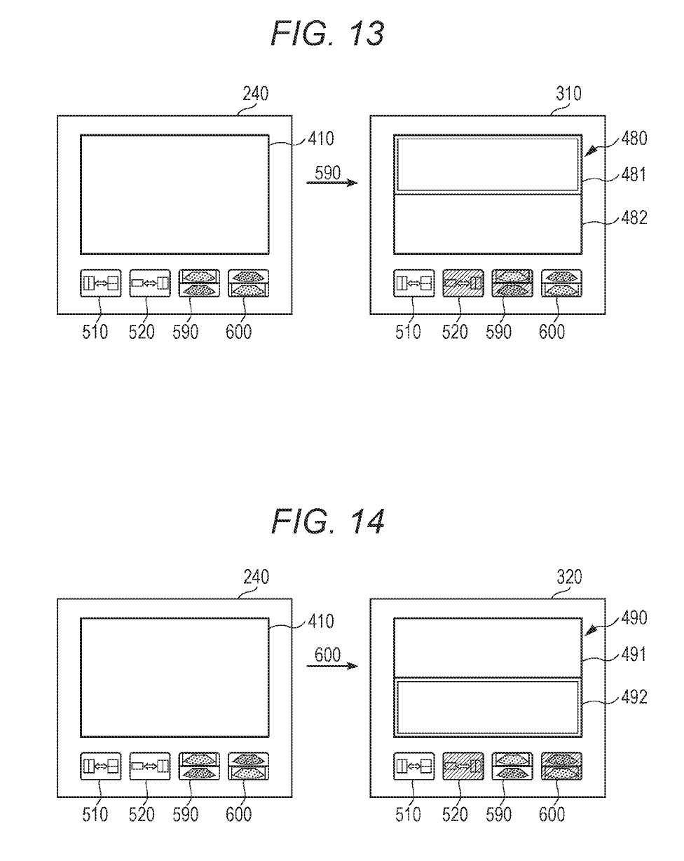

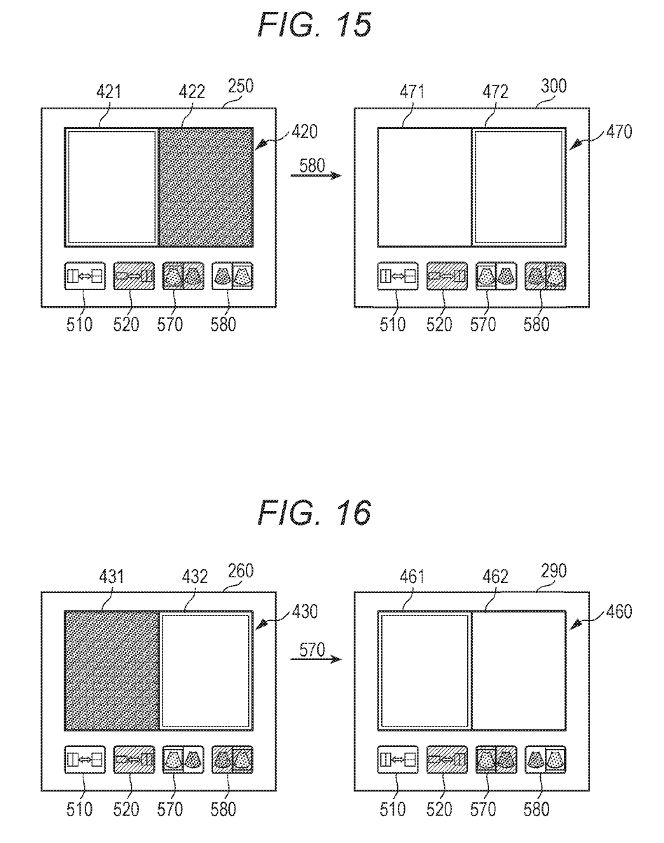

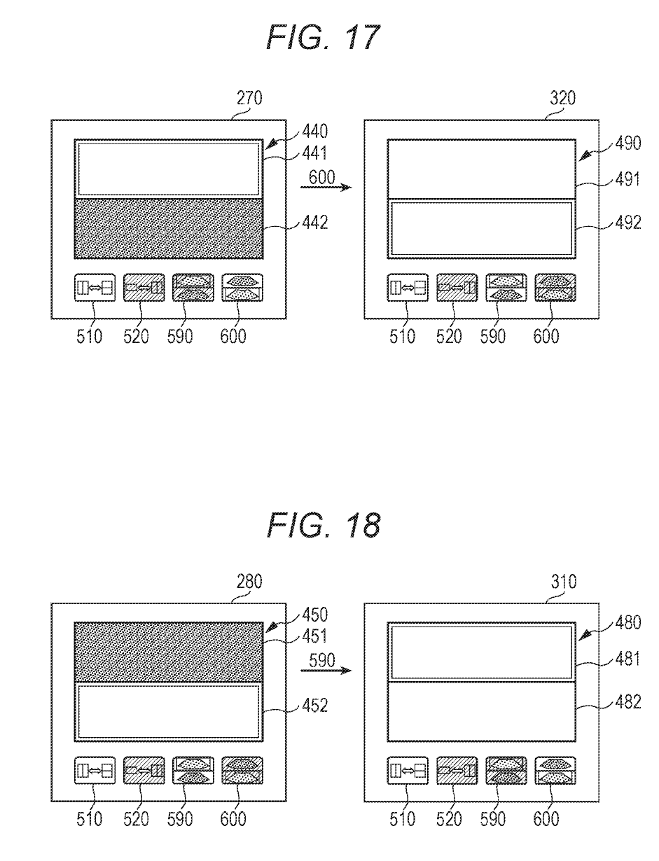

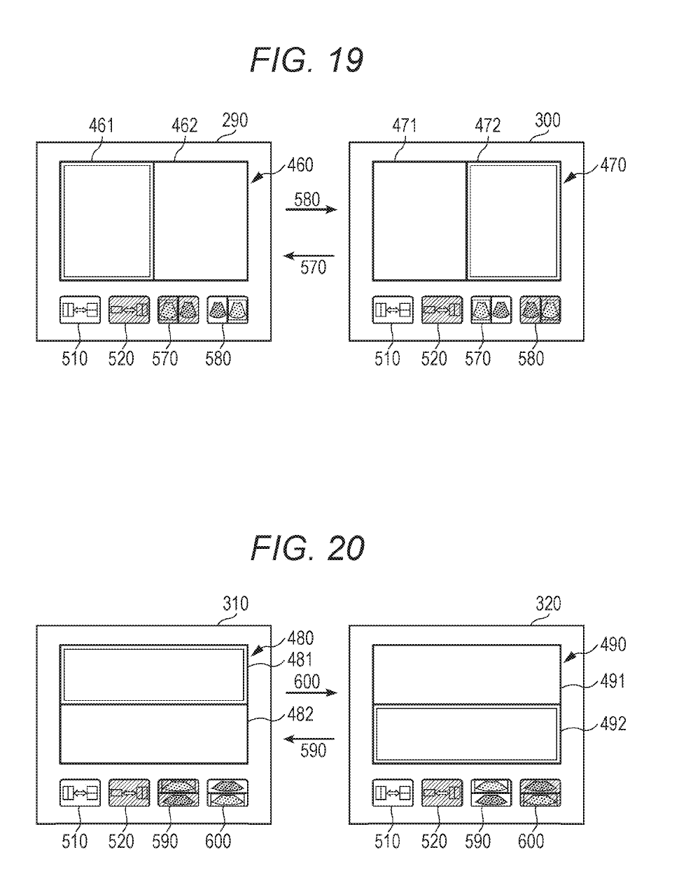

FIG. 11 is a view showing a screen transition between the display screen 230 and a display screen 290. FIG. 12 is a view showing a screen transition between the display screen 230 and a display screen 300. FIG. 13 is a view showing a screen transition between the display screen 240 and a display screen 310. FIG. 14 is a view showing a screen transition between the display screen 240 and a display screen 320. FIG. 15 is a view showing a screen transition between the display screen 250 and the display screen 300. FIG. 16 is a view showing a screen transition between the display screen 260 and the display screen 290. FIG. 17 is a view showing a screen transition between the display screen 270 and the display screen 320. FIG. 18 is a view showing a screen transition between the display screen 280 and the display screen 310. FIG. 19 is a view showing a screen transition between the display screen 290 and the display screen 300. FIG. 20 is a view showing a screen transition between the display screen 310 and the display screen 320.

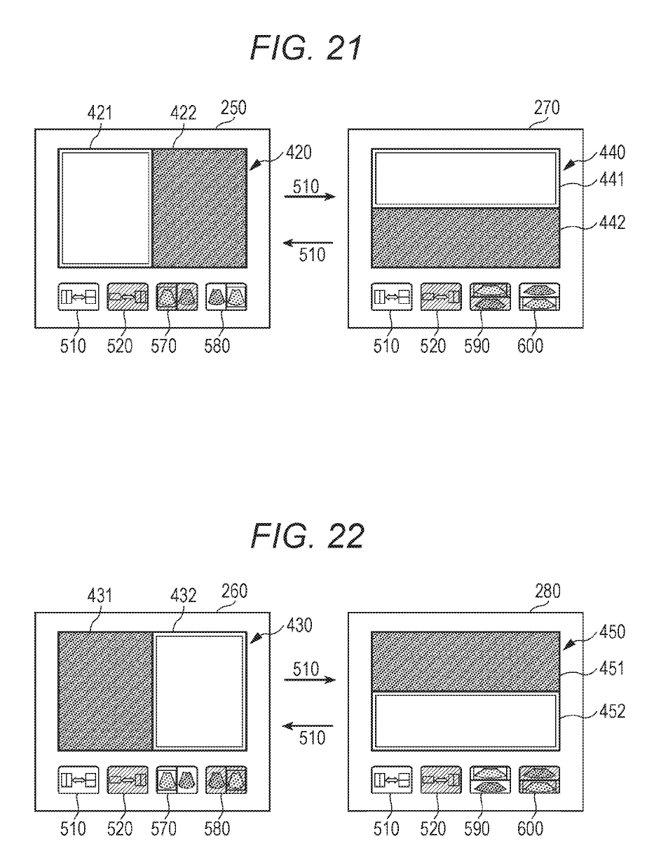

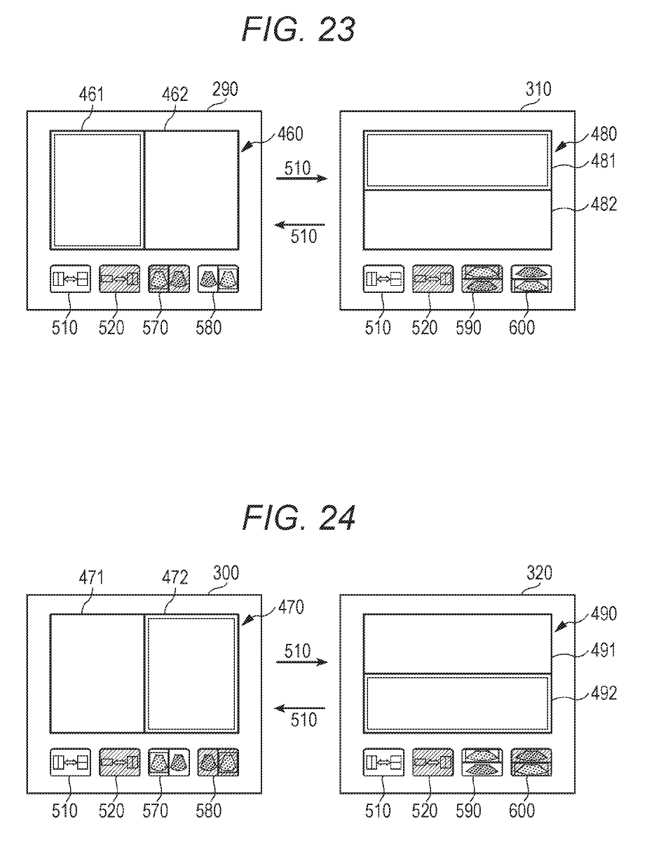

FIG. 21 is a view showing a screen transition between the display screen 250 and the display screen 270. FIG. 22 is a view showing a screen transition between the display screen 260 and the display screen 280. FIG. 23 is a view showing a screen transition between the display screen 290 and the display screen 310. FIG. 24 is a view showing a screen transition between the display screen 300 and the display screen 320.

First, the configuration of the display screen of the ultrasound diagnostic imaging apparatus 1 will be described. As shown in FIG. 3, the display screen (e.g., the display screen 210) of the ultrasound diagnostic imaging apparatus 1 has an ultrasound image region 400 and a button region 500. The ultrasound image region 400 is a display region of an ultrasound image and a display region of one ultrasound image or two horizontal or vertical images. The button region 500 is a display region of display buttons (icons) for transitioning the layout of the ultrasound image in the ultrasound image region 400 and the layout of the display buttons arranged in the button region 500.

The display buttons in the button region 500 are described as ones accepting a touch input through the touch panel 107b, but are not limited thereto. The display buttons may be configured to accept a click input by moving a pointer on the display screen by the operation device of the operation input unit 101. Thus, each of the display screens 210 to 320 has the ultrasound image region 400 and the button region 500.

As shown in FIG. 3, the display screen 210 has a single ultrasound image region 410 arranged in the ultrasound image region 400, a button 510 as a third button, a button 520 as a second button, and buttons 530 and 540 as first buttons, which are arranged in the button region 500. The single ultrasound image region 410 is a display region of one ultrasound image, and, for example, a live image as a real time ultrasound image is displayed.

The button 510 is a display button which accepts inputs as to change the layout of the display buttons in the button region 500 from horizontal to vertical in a single display screen having one ultrasound image region displayed in the ultrasound image region 400 or as to change the layout of the display buttons from vertical to horizontal, and as to change the layout of display images from horizontal to vertical in a dual display screen having two images (an ultrasound image, a blank image) or as to change the layout of the display images from vertical to horizontal. The button 520 is a display button which accepts an input of switching instruction as to whether or not to retain the cine-image data, which is stored in the cine-memory 110 before a transition from the single display screen into the dual display screen in the ultrasound image region 400, after the transition (whether to divide one memory region 110a of the cine-memory 110 into the two memory regions 110b and 110c in advance before the transition).

The button 530 is a display button which indicates a transition into a dual display screen having an active ultrasound image region on the left side and a blank image region on the right side and accepts an input of the transition instruction. The button 540 is a display button which indicates a transition into a dual display screen having a blank image region on the left side and an active ultrasound image region on the right side and accepts an input of the transition instruction.

The display screen 220 has the single ultrasound image region 410 arranged in the ultrasound image region 400, the buttons 510 and 520, and buttons 550 and 560 as first buttons, which are arranged in the button region 500.

The button 550 is a display button which indicates a transition into a dual display screen having an active ultrasound image region on the upper side and a blank image region on the lower side and accepts an input of the transition instruction. The button 560 is a display button which indicates a transition into a dual display screen having a blank image region on the upper side and an active ultrasound image region on the lower side and accepts an input of the transition instruction.

When a touch input is performed on the button 510 while the display screen 210 is displayed, the display screen 210 is transitioned into the display screen 220. Moreover, when a touch input is performed on the button 510 while the display screen 220 is displayed, the display screen 220 is transitioned into the display screen 210. That is, only the button region 500 is changed in the screen transition in FIG. 3. Furthermore, while the display screens 210 and 220 are displayed, one memory region 110a is set in the cine-memory 110, and the cine-image data of the live image displayed in the single ultrasound image region 410 is automatically stored in the memory region 110a. Since the data stored in the memory region 110a is cleared upon the transition from the single display screen into the dual display screen, the buttons 530 to 560 of the display screens 210 and 220 indicate that the cine-image data stored in the cine-memory 110 before the transition from the single display screen into the dual display screen is not retained after the transition.

As shown in FIG. 4, the display screen 230 has the single ultrasound image region 410 arranged in the ultrasound image region 400, the buttons 510 and 520, and buttons 570 and 580 as first buttons, which are arranged in the button region 500.

The button 570 is a display button which indicates a transition into a dual display screen having an active ultrasound image region on the left side and an inactive ultrasound image region on the right side and accepts an input of the transition instruction. The button 580 is a display button which indicates a transition into a dual display screen having an inactive ultrasound image region on the left side and an active ultrasound image region on the right side and accepts an input of the transition instruction.

The display screen 240 has the single ultrasound image region 410 arranged in the ultrasound image region 400, the buttons 510 and 520, and buttons 590 and 600 as first buttons, which are arranged in the button region 500. The button 590 is a display button which indicates a transition into a dual display screen having an active ultrasound image region on the upper side and an inactive ultrasound image region on the lower side and accepts an input of the transition instruction. The button 600 is a display button which indicates a transition into a dual display screen having an inactive ultrasound image region on the upper side and an active ultrasound image region on the lower side and accepts an input of the transition instruction.

When a touch input is performed on the button 510 while the display screen 230 is displayed, the display screen 230 is transitioned into the display screen 240. Moreover, when a touch input is performed on the button 510 while the display screen 240 is displayed, the display screen 240 is transitioned into the display screen 230. That is, only the button region 500 is changed in the screen transition in FIG. 4. Furthermore, while the display screens 230 and 240 are displayed, the two memory regions 110b and 110c, each storing up to 250 frames, are set in the cine-memory 110, and the cine-image data of the live image displayed in the single ultrasound image region 410 is stored in the memory region 110b or 110c. Since the data stored in the memory region 110b or 110c is retained upon the transition from the single display screen into the dual display screen, the buttons 570 to 600 of the display screens 230 and 240 indicate that the cine-image data stored in the cine-memory 110 before the transition from the single display screen into the dual display screen is retained after the transition.

As shown in FIG. 5, when a touch input is performed on the button 520 while the display screen 210 is displayed, the display screen 210 is transitioned into the display screen 230. Moreover, when a touch input is performed on the button 520 while the display screen 230 is displayed, the display screen 230 is transitioned into the display screen 210. That is, only the button region 500 is changed in the screen transition in FIG. 5. Thus, the button 520 accepts a switching input as to whether or not to retain the cine-image data, which is stored in the cine-memory 110 before the transition from the single display screen to the dual display screen, after the transition.

As shown in FIG. 6, when a touch input is performed on the button 520 while the display screen 220 is displayed, the display screen 220 is transitioned into the display screen 240. Moreover, when a touch input is performed on the button 520 while the display screen 240 is displayed, the display screen 240 is transitioned into the display screen 220. That is, only the button region 500 is changed in the screen transition in FIG. 6.

As shown in FIG. 7, the display screen 250 has a dual image region 420 arranged in the ultrasound image region 400 and the buttons 510, 520, 570 and 580 arranged in the button region 500. However, the buttons 520 and 570 of the display screen 250 are displayed as input disabled states (grayed out), and the touch input is disabled.

The dual image region 420 has two image regions, one active ultrasound image region 421 arranged on the left side and a blank image region 422 which is arranged on the right side and is blank where an ultrasound image is not displayed. In the ultrasound image region 421, for example, a live image as a real time ultrasound image is displayed.

When a touch input is performed on the button 530 while the display screen 210 is displayed, the display screen 210 is transitioned into the display screen 250. For example, while the display screen 210 is displayed, the live image is displayed in the single ultrasound image region 410, and the cine-image data of the live image is stored in the memory region 110a of the cine-memory 110. When the touch input is performed on the button 530, the cine-image data stored in the memory region 110a of the cine-memory 110 is cleared as well as the memory region 110a is divided into the memory regions 110b and 110c, and the cine-image data of the live image started to be displayed in the ultrasound image region 421 is started to be stored in the memory region 110b. The memory region 110c is kept empty, corresponding to the blank image region 422. Thus, the button 530 indicates that the cine-image data stored in the cine-memory 110 before a transition from the single display screen into the dual display screen is not retained after the transition. Note that the cine-image data of the live image displayed in the ultrasound image region 421 may be stored in the memory region 110c and the memory region 110b may be empty, corresponding to the blank image region 422.

As shown in FIG. 8, the display screen 260 has a dual image region 430 arranged in the ultrasound image region 400 and the buttons 510, 520, 570 and 580 arranged in the button region 500. However, the buttons 520 and 580 of the display screen 260 are displayed as input disabled states, and the touch input is disabled.

The dual image region 430 has two image regions, a blank image region 431 which is arranged on the left side and is blank where an ultrasound image is not displayed and one active ultrasound image region 432 arranged on the right side. In the ultrasound image region 432, for example, a live image as a real time ultrasound image is displayed.

When a touch input is performed on the button 540 while the display screen 210 is displayed, the display screen 210 is transitioned into the display screen 260. For example, while the display screen 210 is displayed, the live image is displayed in the single ultrasound image region 410, and the cine-image data of the live image is stored in the memory region 110a of the cine-memory 110. When the touch input is performed on the button 540, the cine-image data stored in the memory region 110a of the cine-memory 110 is cleared as well as the memory region 110a is divided into the memory regions 110b and 110c, and the cine-image data of the live image started to be displayed in the ultrasound image region 432 is started to be stored in the memory region 110c. The memory region 110b is kept empty, corresponding to the blank image region 431. Thus, the button 540 indicates that the cine-image data stored in the cine-memory 110 before a transition from the single display screen into the dual display screen is not retained after the transition. Note that the cine-image data of the live image displayed in the ultrasound image region 432 may be stored in the memory region 110b and the memory region 110c may be empty, corresponding to the blank image region 431.

As shown in FIG. 9, the display screen 270 has a dual image region 440 arranged in the ultrasound image region 400 and the buttons 510, 520, 590 and 600 arranged in the button region 500. However, the buttons 520 and 590 of the display screen 270 are displayed as input disabled states, and the touch input is disabled.

The dual image region 440 has two image regions, one active ultrasound image region 441 arranged on the upper side and a blank image region 442 which is arranged on the lower side and is blank where an ultrasound image is not displayed. In the ultrasound image region 441, for example, a live image as a real time ultrasound image is displayed.

When a touch input is performed on the button 550 while the display screen 220 is displayed, the display screen 220 is transitioned into the display screen 270. For example, while the display screen 220 is displayed, the live image is displayed in the single ultrasound image region 410, and the cine-image data of the live image is stored in the memory region 110a of the cine-memory 110. When the touch input is performed on the button 550, the cine-image data stored in the memory region 110a of the cine-memory 110 is cleared as well as the memory region 110a is divided into the memory regions 110b and 110c, and the cine-image data of the live image started to be displayed in the ultrasound image region 441 is started to be stored in the memory region 110b. The memory region 110c is kept empty, corresponding to the blank image region 442. Thus, the button 550 indicates that the cine-image data stored in the cine-memory 110 before a transition from the single display screen into the dual display screen is not retained after the transition. Note that the cine-image data of the live image displayed in the ultrasound image region 441 may be stored in the memory region 110c and the memory region 110b may be empty, corresponding to the blank image region 442.

As shown in FIG. 10, the display screen 280 has a dual image region 450 arranged in the ultrasound image region 400 and the buttons 510, 520, 590 and 600 arranged in the button region 500. However, the buttons 520 and 600 of the display screen 280 are displayed as input disabled states, and the touch input is disabled.

The dual image region 450 has two image regions, a blank image region 451 which is arranged on the upper side and is blank where an ultrasound image is not displayed and one active ultrasound image region 452 arranged on the lower side. In the ultrasound image region 452, for example, a live image as a real time ultrasound image is displayed.

When a touch input is performed on the button 560 while the display screen 220 is displayed, the display screen 220 is transitioned into the display screen 280. For example, while the display screen 220 is displayed, the live image is displayed in the single ultrasound image region 410, and the cine-image data of the live image is stored in the memory region 110a of the cine-memory 110. When the touch input is performed on the button 560, the cine-image data stored in the memory region 110a of the cine-memory 110 is cleared as well as the memory region 110a is divided into the memory regions 110b and 110c, and the cine-image data of the live image started to be displayed in the ultrasound image region 452 is started to be stored in the memory region 110c. The memory region 110b is kept empty, corresponding to the blank image region 451. Thus, the button 560 indicates that the cine-image data stored in the cine-memory 110 before a transition from the single display screen into the dual display screen is not retained after the transition. Note that the cine-image data of the live image displayed in the ultrasound image region 452 may be stored in the memory region 110b and the memory region 110c may be empty, corresponding to the blank image region 451.

As shown in FIG. 11, the display screen 290 has a dual image region 460 arranged in the ultrasound image region 400 and the buttons 510, 520, 570 and 580 arranged in the button region 500. However, the buttons 520 and 570 of the display screen 290 are displayed as input disabled states, and the touch input is disabled.

The dual image region 460 has two image regions, one active ultrasound image region 461 arranged on the left side and one inactive ultrasound image region 462 arranged on the right side. In the ultrasound image region 461, for example, a live image as a real time ultrasound image is displayed. In the ultrasound image region 462, for example, a cine-image of cine-image data stored before a screen transition into the display screen 290 is displayed.

When a touch input is performed on the button 570 while the display screen 230 is displayed, the display screen 230 is transitioned into the display screen 290. For example, while the display screen 230 is displayed, the live image is displayed in the single ultrasound image region 410, and the cine-image data of the live image is stored in the memory region 110b of the cine-memory 110. When the touch input is performed on the button 570, the cine-image data of the live image started to be displayed in the ultrasound image region 461 is started to be stored in the memory region 110c which has been empty. The cine-image data stored in the memory region 110b is displayed in the ultrasound image region 462. For example, in an initial state, a still image of the last frame of the cine-image data before a screen transition is displayed in the ultrasound image region 462. Thus, the button 570 indicates that the cine-image data stored in the cine-memory 110 before a transition from the single display screen into the dual display screen is retained after the transition. Note that the cine-image data of the live image displayed in the single ultrasound image region 410 may be stored in the memory region 110c and the cine-image data of the live image displayed in the ultrasound image region 461 may be stored in the memory region 110b.

As shown in FIG. 12, the display screen 300 has a dual image region 470 arranged in the ultrasound image region 400 and the buttons 510, 520, 570 and 580 arranged in the button region 500. However, the buttons 520 and 580 of the display screen 300 are displayed as input disabled states, and the touch input is disabled.

The dual image region 470 has two image regions, one inactive ultrasound image region 471 arranged on the left side and one active ultrasound image region 472 arranged on the right side. In the ultrasound image region 471, for example, a cine-image of cine-image data stored before a screen transition into the display screen 300 is displayed. In the ultrasound image region 472, for example, alive image as a real time ultrasound image is displayed.

When a touch input is performed on the button 580 while the display screen 230 is displayed, the display screen 230 is transitioned into the display screen 300. For example, while the display screen 230 is displayed, the live image is displayed in the single ultrasound image region 410, and the cine-image data of the live image is stored in the memory region 110b of the cine-memory 110. When the touch input is performed on the button 580, the cine-image data of the live image started to be displayed in the ultrasound image region 472 is started to be stored in the memory region 110c which has been empty. The cine-image data stored in the memory region 110b is displayed in the ultrasound image region 471. For example, in an initial state, a still image of the last frame of the cine-image data before a screen transition is displayed in the ultrasound image region 471. Thus, the button 580 indicates that the cine-image data stored in the cine-memory 110 before a transition from the single display screen into the dual display screen is retained after the transition. Note that the cine-image data of the live image displayed in the single ultrasound image region 410 may be stored in the memory region 110c and the cine-image data of the live image displayed in the ultrasound image region 472 may be stored in the memory region 110b.

As shown in FIG. 13, the display screen 310 has a dual image region 480 arranged in the ultrasound image region 400 and the buttons 510, 520, 590 and 600 arranged in the button region 500. However, the buttons 520 and 590 of the display screen 310 are displayed as input disabled states, and the touch input is disabled.

The dual image region 480 has two image regions, one active ultrasound image region 481 arranged on the upper side and one inactive ultrasound image region 482 arranged on the lower side. In the ultrasound image region 481, for example, a live image as a real time ultrasound image is displayed. In the ultrasound image region 482, for example, a cine-image of cine-image data stored before a screen transition into the display screen 310 is displayed.

When a touch input is performed on the button 590 while the display screen 240 is displayed, the display screen 240 is transitioned into the display screen 310. For example, while the display screen 240 is displayed, the live image is displayed in the single ultrasound image region 410, and the cine-image data of the live image is stored in the memory region 110b of the cine-memory 110. When the touch input is performed on the button 590, the cine-image data of the live image started to be displayed in the ultrasound image region 481 is started to be stored in the memory region 110c which has been empty. The cine-image data stored in the memory region 110b is displayed in the ultrasound image region 482. For example, in an initial state, a still image of the last frame of the cine-image data before a screen transition is displayed in the ultrasound image region 482. Thus, the button 590 indicates that the cine-image data stored in the cine-memory 110 before a transition from the single display screen into the dual display screen is retained after the transition. Note that the cine-image data of the live image displayed in the single ultrasound image region 410 may be stored in the memory region 110c and the cine-image data of the live image displayed in the ultrasound image region 481 may be stored in the memory region 110b.

As shown in FIG. 14, the display screen 320 has a dual image region 490 arranged in the ultrasound image region 400 and the buttons 510, 520, 590 and 600 arranged in the button region 500. However, the buttons 520 and 600 of the display screen 320 are displayed as input disabled states, and the touch input is disabled.

The dual image region 490 has two image regions, one inactive ultrasound image region 491 arranged on the upper side and one active ultrasound image region 492 arranged on the lower side. In the ultrasound image region 491, for example, a cine-image of cine-image data stored before a screen transition into the display screen 320 is displayed. In the ultrasound image region 492, for example, alive image as a real time ultrasound image is displayed.

When a touch input is performed on the button 600 while the display screen 240 is displayed, the display screen 240 is transitioned into the display screen 320. For example, while the display screen 240 is displayed, the live image is displayed in the single ultrasound image region 410, and the cine-image data of the live image is stored in the memory region 110b of the cine-memory 110. When the touch input is performed on the button 600, the cine-image data of the live image started to be displayed in the ultrasound image region 492 is started to be stored in the memory region 110c which has been empty. The cine-image data stored in the memory region 110b is displayed in the ultrasound image region 491. For example, in an initial state, a still image of the last frame of the cine-image data before a screen transition is displayed in the ultrasound image region 491. Thus, the button 600 indicates that the cine-image data stored in the cine-memory 110 before a transition from the single display screen into the dual display screen is retained after the transition. Note that the cine-image data of the live image displayed in the single ultrasound image region 410 may be stored in the memory region 110c and the cine-image data of the live image displayed in the ultrasound image region 492 may be stored in the memory region 110b.

As shown in FIG. 15, when a touch input is performed on the button 580 while the display screen 250 is displayed, the display screen 250 is transitioned into the display screen 300. For example, while the display screen 250 is displayed, the live image is displayed in the ultrasound image region 421, and the cine-image data of the live image is stored in the memory region 110b of the cine-memory 110. When the touch input is performed on the button 580, the cine-image data of the live image started to be displayed in the ultrasound image region 472 is started to be stored in the memory region 110c which has been empty. The cine-image data stored in the memory region 110b is displayed in the ultrasound image region 471. Note that the cine-image data of the live image displayed in the ultrasound image region 421 may be stored in the memory region 110c and the cine-image data of the live image displayed in the ultrasound image region 472 may be stored in the memory region 110b.

As shown in FIG. 16, when a touch input is performed on the button 570 while the display screen 260 is displayed, the display screen 260 is transitioned into the display screen 290. For example, while the display screen 260 is displayed, the live image is displayed in the ultrasound image region 432, and the cine-image data of the live image is stored in the memory region 110c of the cine-memory 110. When the touch input is performed on the button 570, the cine-image data of the live image started to be displayed in the ultrasound image region 461 is started to be stored in the memory region 110b which has been empty. The cine-image data stored in the memory region 110c is displayed in the ultrasound image region 462. Note that the cine-image data of the live image displayed in the ultrasound image region 432 may be stored in the memory region 110b and the cine-image data of the live image displayed in the ultrasound image region 461 may be stored in the memory region 110c.

As shown in FIG. 17, when a touch input is performed on the button 600 while the display screen 270 is displayed, the display screen 270 is transitioned into the display screen 320. For example, while the display screen 270 is displayed, the live image is displayed in the ultrasound image region 441, and the cine-image data of the live image is stored in the memory region 110b of the cine-memory 110. When the touch input is performed on the button 600, the cine-image data of the live image started to be displayed in the ultrasound image region 492 is started to be stored in the memory region 110c which has been empty. The cine-image data stored in the memory region 110b is displayed in the ultrasound image region 491. Note that the cine-image data of the live image displayed in the ultrasound image region 441 may be stored in the memory region 110c and the cine-image data of the live image displayed in the ultrasound image region 492 may be stored in the memory region 110b.

As shown in FIG. 18, when a touch input is performed on the button 590 while the display screen 280 is displayed, the display screen 280 is transitioned into the display screen 310. For example, while the display screen 280 is displayed, the live image is displayed in the ultrasound image region 452, and the cine-image data of the live image is stored in the memory region 110c of the cine-memory 110. When the touch input is performed on the button 590, the cine-image data of the live image started to be displayed in the ultrasound image region 481 is started to be stored in the memory region 110b which has been empty. The cine-image data stored in the memory region 110c is displayed in the ultrasound image region 482. Note that the cine-image data of the live image displayed in the ultrasound image region 452 may be stored in the memory region 110b and the cine-image data of the live image displayed in the ultrasound image region 481 may be stored in the memory region 110c.

As shown in FIG. 19, when a touch input is performed on the button 580 while the display screen 290 is displayed, the display screen 290 is transitioned into the display screen 300. For example, while the display screen 290 is displayed, the live image is displayed in the ultrasound image region 461, and the cine-image data of the live image is stored in the memory region 110b of the cine-memory 110. The cine-image data stored in the memory region 110c is displayed in the ultrasound image region 462. When the touch input is performed on the button 580, the cine-image data of the live image started to be displayed in the ultrasound image region 472 is started to be stored in (overwriting) the memory region 110c. The cine-image data stored in the memory region 110b is displayed in the ultrasound image region 471.

When a touch input is performed on the button 570 while the display screen 300 is displayed, the display screen 300 is transitioned into the display screen 290. For example, while the display screen 300 is displayed, the live image is displayed in the ultrasound image region 472, and the cine-image data of the live image is stored in the memory region 110c of the cine-memory 110. The cine-image data stored in the memory region 110b is displayed in the ultrasound image region 471. When the touch input is performed on the button 570, the cine-image data of the live image started to be displayed in the ultrasound image region 461 is started to be stored in (overwriting) the memory region 110b. The cine-image data stored in the memory region 110c is displayed in the ultrasound image region 462. Note that the cine-image data of the live image displayed in the ultrasound image region 461 may be stored in the memory region 110c and the cine-image data of the live image displayed in the ultrasound image region 472 may be stored in the memory region 110b.

As shown in FIG. 20, when a touch input is performed on the button 600 while the display screen 310 is displayed, the display screen 310 is transitioned into the display screen 320. For example, while the display screen 310 is displayed, the live image is displayed in the ultrasound image region 481, and the cine-image data of the live image is stored in the memory region 110b of the cine-memory 110. The cine-image data stored in the memory region 110c is displayed in the ultrasound image region 482. When the touch input is performed on the button 600, the cine-image data of the live image started to be displayed in the ultrasound image region 492 is started to be stored in (overwriting) the memory region 110c. The cine-image data stored in the memory region 110b is displayed in the ultrasound image region 491.

When a touch input is performed on the button 590 while the display screen 320 is displayed, the display screen 320 is transitioned into the display screen 310. For example, while the display screen 320 is displayed, the live image is displayed in the ultrasound image region 492, and the cine-image data of the live image is stored in the memory region 110c of the cine-memory 110. The cine-image data stored in the memory region 110b is displayed in the ultrasound image region 491. When the touch input is performed on the button 590, the cine-image data of the live image started to be displayed in the ultrasound image region 481 is started to be stored in (overwriting) the memory region 110b. The cine-image data stored in the memory region 110c is displayed in the ultrasound image region 482. Note that the cine-image data of the live image displayed in the ultrasound image region 481 may be stored in the memory region 110c and the cine-image data of the live image displayed in the ultrasound image region 492 maybe stored in the memory region 110b.

As shown in FIG. 21, when a touch input is performed on the button 510 while the display screen 250 is displayed, the display screen 250 is transitioned into the display screen 270. For example, while the display screen 250 is displayed, the live image is displayed in the ultrasound image region 421, and the cine-image data of the live image is stored in the memory region 110b of the cine-memory 110. When the touch input is performed on the button 510, the cine-image data of the live image started to be displayed in the ultrasound image region 441 is continued to be stored in the memory region 110b.

When a touch input is performed on the button 510 while the display screen 270 is displayed, the display screen 270 is transitioned into the display screen 250. For example, while the display screen 270 is displayed, the live image is displayed in the ultrasound image region 441, and the cine-image data of the live image is stored in the memory region 110b of the cine-memory 110. When the touch input is performed on the button 510, the cine-image data of the live image started to be displayed in the ultrasound image region 421 is continued to be stored in the memory region 110b. Note that the cine-image data of the live image displayed in the ultrasound image region 421 may be stored in the memory region 110c and the cine-image data of the live image displayed in the ultrasound image region 441 may be stored in the memory region 110c.

As shown in FIG. 22, when a touch input is performed on the button 510 while the display screen 260 is displayed, the display screen 260 is transitioned into the display screen 280. For example, while the display screen 260 is displayed, the live image is displayed in the ultrasound image region 432, and the cine-image data of the live image is stored in the memory region 110c of the cine-memory 110. When the touch input is performed on the button 510, the cine-image data of the live image started to be displayed in the ultrasound image region 452 is continued to be stored in the memory region 110c.

When a touch input is performed on the button 510 while the display screen 280 is displayed, the display screen 280 is transitioned into the display screen 260. For example, while the display screen 280 is displayed, the live image is displayed in the ultrasound image region 452, and the cine-image data of the live image is stored in the memory region 110c of the cine-memory 110. When the touch input is performed on the button 510, the cine-image data of the live image started to be displayed in the ultrasound image region 432 is continued to be stored in the memory region 110c. Note that the cine-image data of the live image displayed in the ultrasound image region 432 may be stored in the memory region 110b and the cine-image data of the live image displayed in the ultrasound image region 452 may be stored in the memory region 110b.

As shown in FIG. 23, when a touch input is performed on the button 510 while the display screen 290 is displayed, the display screen 290 is transitioned into the display screen 310. For example, while the display screen 290 is displayed, the live image is displayed in the ultrasound image region 461, and the cine-image data of the live image is stored in the memory region 110b of the cine-memory 110. The cine-image data stored in the memory region 110c is displayed in the ultrasound image region 462. When the touch input is performed on the button 510, the cine-image data of the live image started to be displayed in the ultrasound image region 481 is continued to be stored in the memory region 110b. The cine-image data stored in the memory region 110c is displayed in the ultrasound image region 482.

When a touch input is performed on the button 510 while the display screen 310 is displayed, the display screen 310 is transitioned into the display screen 290. For example, while the display screen 310 is displayed, the live image is displayed in the ultrasound image region 481, and the cine-image data of the live image is stored in the memory region 110b of the cine-memory 110. The cine-image data stored in the memory region 110c is displayed in the ultrasound image region 482. When the touch input is performed on the button 510, the cine-image data of the live image started to be displayed in the ultrasound image region 461 is continued to be stored in the memory region 110b. The cine-image data stored in the memory region 110c is displayed in the ultrasound image region 462. Note that the cine-image data of the live image displayed in the ultrasound image region 461 may be stored in the memory region 110c and the cine-image data of the live image displayed in the ultrasound image region 481 may be stored in the memory region 110c.

As shown in FIG. 24, when a touch input is performed on the button 510 while the display screen 300 is displayed, the display screen 300 is transitioned into the display screen 320. For example, while the display screen 300 is displayed, the live image is displayed in the ultrasound image region 472, and the cine-image data of the live image is stored in the memory region 110c of the cine-memory 110. The cine-image data stored in the memory region 110b is displayed in the ultrasound image region 471. When the touch input is performed on the button 510, the cine-image data of the live image started to be displayed in the ultrasound image region 492 is continued to be stored in the memory region 110c. The cine-image data stored in the memory region 110b is displayed in the ultrasound image region 491.

When a touch input is performed on the button 510 while the display screen 320 is displayed, the display screen 320 is transitioned into the display screen 300. For example, while the display screen 320 is displayed, the live image is displayed in the ultrasound image region 492, and the cine-image data of the live image is stored in the memory region 110c of the cine-memory 110. The cine-image data stored in the memory region 110b is displayed in the ultrasound image region 491. When the touch input is performed on the button 510, the cine-image data of the live image started to be displayed in the ultrasound image region 472 is continued to be stored in the memory region 110c. The cine-image data stored in the memory region 110b is displayed in the ultrasound image region 471. Note that the cine-image data of the live image displayed in the ultrasound image region 472 may be stored in the memory region 110b and the cine-image data of the live image displayed in the ultrasound image region 492 may be stored in the memory region 110b.

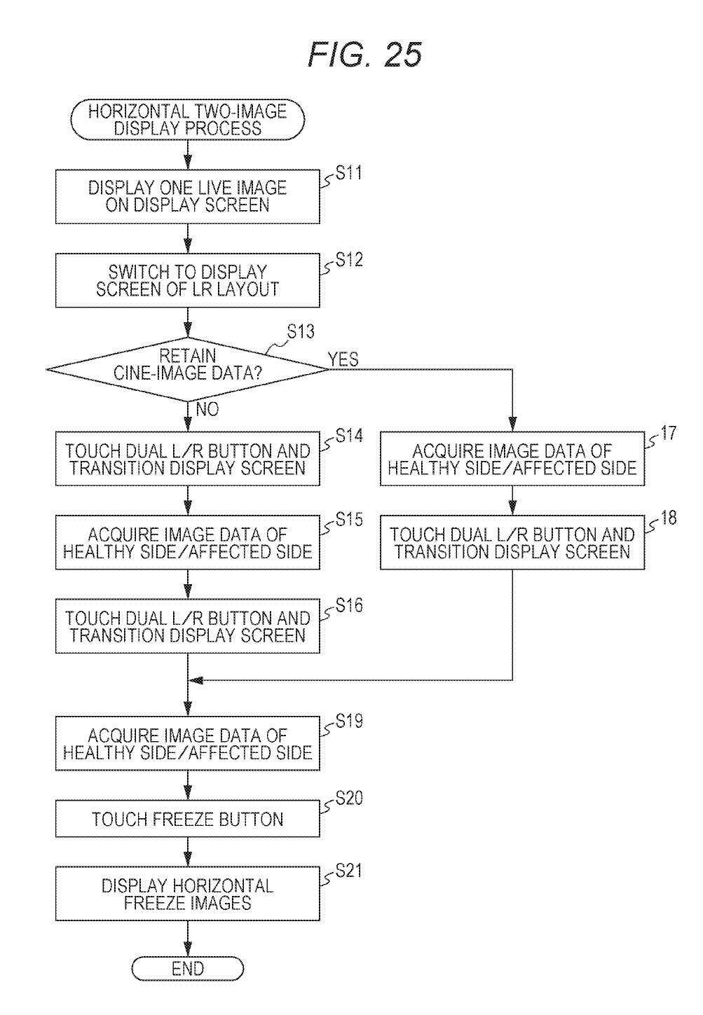

Next, a horizontal two-image display process using the screen transitions described above will be described with reference to FIG. 25. FIG. 25 is a flowchart showing the horizontal two-image display process. As one example, the horizontal two-image display process is a process which generates ultrasound image data of a healthy part side and an affected part side of a test subject of a patient having the affected part in one of a pair of parts such as hands, arms or legs and displays the freeze images thereof as two horizontal images on the screen.

First, the controller 108 generates image data of the display screen 210, 220, 230 or 240, displays the data on the display unit 107a, controls the transmission unit 102, the reception unit 103, the image generation unit 104, the image processing unit 105, the DSC 106 and the operation display unit 107 to sequentially generate real time ultrasound image data (B-mode image data) and displays the data as a live image in the single ultrasound image region 410 of the display screen of the display unit 107a as well as sequentially stores the generated B-mode image data as cine-image data in the memory region 110a, 110b or 110c of the cine-memory 110 (step S11). While the display screen 210 or 230 is displayed, the cine-image data of the live image is stored in the memory region 110a. While the display screen 220 or 240 is displayed, the cine-image data of the live image is stored in the memory region 110b or 110c.

Then, when the display screen being displayed is the display screen 210 or 230, the controller 108 leaves the display screen as it is. When the display screen being displayed is the display screen 220 or 240, the controller 108 accepts a touch input on the button 510 from an examiner through the touch panel 107b and, according to the touch input on the button 510, transitions the display screen 220 being displayed into the display screen 210 or transitions the display screen 240 being displayed into the display screen 230 to display on the display unit 107a (step S12). Moreover, in the step S12, the controller 108 accepts a touch input on the button 520 from the examiner and, according to the touch input, transitions the display screen 210 into the display screen 230 or transitions the display screen 230 into the display screen 210.

Then, the controller 108 determines whether or not to retain the cine-image data, which is stored in the cine-memory 110 before a transition from a display screen having one ultrasound image into a display screen having two images, after the transition according to the buttons 530 to 580 of the display screen 210 or 230 being displayed (step S13). When the display screen being displayed is the display screen 210 having the buttons 530 and 540, the cine-image data stored in the cine-memory 110 before the transition is determined not to be retained after the transition. When the display screen being displayed is the display screen 230 having the buttons 570 and 580, the cine-image data stored in the cine-memory 110 before the transition is determined to be retained after the transition.

When the cine-image data stored in the cine-memory 110 before the transition is not retained (step S13; NO), the controller 108 accepts a touch input on the button 530 or 540 as a dual L/R button from the examiner through the touch panel 107b, transitions the display screen 210 into the display screen 250 or 260 according to the touch input button and clears the cine-memory 110 to be divided into the memory regions 110b and 110c (step S14). The live image is displayed in the ultrasound image region 421 in the display screen 250 or the ultrasound image region 432 in the display screen 260. Herein, the examiner presses the ultrasound probe 1b against a part of the test subject, which is scanned first. For example, to ultimately set the left side as the healthy part side and the right side as the affected part side, the examiner presses the ultrasound probe 1b against a site of the healthy part side when the display screen 250 with the active ultrasound image region 421 on the left side is being displayed, and presses the ultrasound probe 1b against a site of the affected part side when the display screen 260 with the active ultrasound image region 432 on the right side is being displayed.

Then, the controller 108 generates ultrasound image data of the site of the healthy part side or the affected part side, against which the ultrasound probe 1b is pressed in the step S14, displays the data as a live image in the ultrasound image region 421 or 432 and stores the ultrasound image data as cine-image data in the memory region 110b or 110c of the cine-memory 110 (step S15).

Then, the controller 108 accepts a touch input on the button 570 or 580, which is a dual L/R button and not disabled, from the examiner through the touch panel 107b, transitions the display screen 250 or 260 into the display screen 300 or 290 according to the touch input button, starts to display the live ultrasound image data started to be newly scanned in the ultrasound image region 472 or 461 (opposite side of the live image display side until the step S15), stops storing the new cine-image data in, for example, the memory region 110b or 110c of the cine-memory 110 which has last been storing the cine-image data, and starts to store the cine-image data of the live image in the memory region 110b or 110c which has not last been storing the cine-image data (step S16).

In the step S16, while the display screen 290 is displayed, the last frame of the cine-image data stored in the memory region 110b or 110c in the step S15 is displayed as a freeze image in the ultrasound image region 462. Similarly, while the display screen 300 is displayed, the last frame of the cine-image data stored in the memory region 110b or 110c in the step S15 is displayed as a freeze image in the ultrasound image region 471. Moreover, the examiner presses the ultrasound probe 1b against a part of the test subject not scanned in the step S15. For example, the examiner presses the ultrasound probe 1b against a site of the healthy part side when the display screen 290 with the active ultrasound image region 461 on the left side is being displayed, and presses the ultrasound probe 1b against a site of the affected part side when the display screen 300 with the active ultrasound image region 472 on the right side is being displayed.

When the cine-image data stored in the cine-memory 110 before the transition is retained after the transition (step S13; YES), the examiner presses the ultrasound probe 1b in advance against a part of the test subject, which is scanned first. For example, the examiner presses the ultrasound probe 1b against a site of the healthy part side when the display screen 290 with the active ultrasound image region 461 on the left side is displayed next, and presses the ultrasound probe 1b against a site of the affected part side when the display screen 300 with the active ultrasound image region 472 on the right side is displayed next. Herein, the controller 108 generates ultrasound image data of the site of the healthy part side or the affected part side, against which the ultrasound probe 1b is pressed, continues to display the data as a live image in the single ultrasound image region 410 and stores the ultrasound image data as cine-image data in the memory region 110b or 110c of the cine-memory 110 (step S17).

Then, the controller 108 accepts a touch input on the button 570 or 580 as a dual L/R button from the examiner through the touch panel 107b, transitions the display screen 230 into the display screen 290 or 300 according to the touch input button, starts to display the live image in the ultrasound image region 461 or 472, and starts to store the cine-image data of the live image in the memory region 110b or 110c (the memory region not storing the cine-image data in the step S17) of the cine-memory 110 (step S18). In the step S18, while the display screen 290 is displayed, the last frame of the cine-image data stored in the memory region 110b or 110c in the step S17 is displayed as a freeze image in the ultrasound image region 462. Similarly, while the display screen 300 is displayed, the last frame of the cine-image data stored in the memory region 110b or 110c in the step S17 is displayed as a freeze image in the ultrasound image region 471. Moreover, the examiner presses the ultrasound probe 1b against a part of the test subject not scanned in the step S17. For example, the examiner presses the ultrasound probe 1b against a site of the healthy part side when the display screen 290 with the active ultrasound image region 461 on the left side is being displayed, and presses the ultrasound probe 1b against a site of the affected part side when the display screen 300 with the active ultrasound image region 472 on the right side is being displayed.

After the step S16 or S18, the controller 108 generates ultrasound image data of the site of the healthy part side or the affected part side, against which the ultrasound probe 1b is pressed in the step S16 or S18, displays the data as a live image in the ultrasound image region 461 or 472 and starts to store the ultrasound image data as cine-image data in the memory region 110b or 110c (the memory region not storing the cine-image data in the step S15 or S17) of the cine-memory 110 (step S19). Then, the controller 108 accepts a touch input on a freeze button (not illustrated) on the display screen from the examiner through the touch panel 107b (step S20).

Then, the controller 108 stops scanning the ultrasound image and storing the cine-image data according the touch input on the freeze button in the step S20, displays a freeze image of the last frame of the cine-image data of the live image in the ultrasound image region 461 in the display screen 290 or the ultrasound image region 472 in the display screen 300 (step S21) and ends the horizontal two-image display process. The image data of the freeze image is, for example, stored in the memory 109 by the controller 108 according to an instruction input from the examiner through the operation input unit 101.

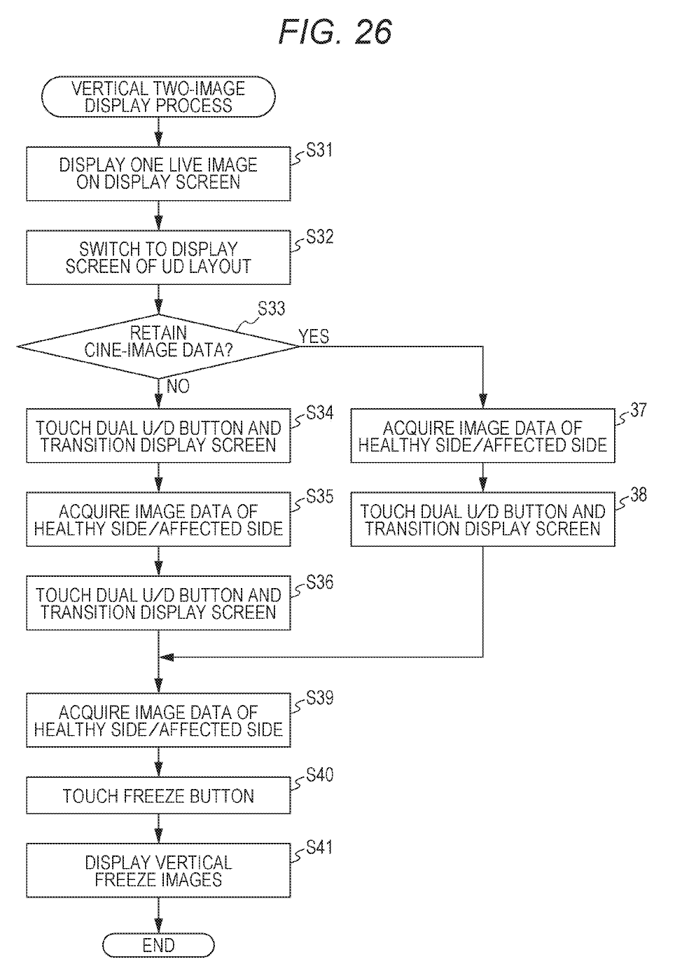

Next, a vertical two-image display process using the aforementioned screen transitions will be described with reference to FIG. 26. FIG. 26 is a flowchart showing the vertical two-image display process. As one example, the vertical two-image display process is a process which generates ultrasound image data of a healthy part side and an affected part side of a test subject of a patient having the affected part in one of a pair of parts and displays the freeze images thereof as two vertical images on the screen.

First, a step S31 is the same as the step S11 in FIG. 25. Then, when the display screen being displayed is the display screen 220 or 240, the controller 108 leaves the display screen as it is. When the display screen being displayed is the display screen 210 or 230, the controller 108 accepts a touch input on the button 510 from the examiner through the touch panel 107b and, according the touch input on the button 510, transitions the display screen 210 being displayed into the display screen 220 or transitions the display screen 230 being displayed into the display screen 240 to display on the display unit 107a (step S32). Moreover, in the step S32, the controller 108 accepts a touch input on the button 520 from the examiner and, according the touch input, transitions the display screen 220 into the display screen 240 or transitions the display screen 240 into the display screen 220.

Then, the controller 108 determines whether or not to retain the cine-image data, which is stored in the cine-memory 110 before a transition from a display screen having one ultrasound image into a display screen having two images, after the transition according to the buttons 550 to 600 of the display screen 220 or 240 being displayed (step S33). When the display screen being displayed is the display screen 220 having the buttons 550 and 560, the cine-image data stored in the cine-memory 110 before the transition is determined not to be retained after the transition. When the display screen being displayed is the display screen 240 having the buttons 590 and 600, the cine-image data stored in the cine-memory 110 before the transition is determined to be retained after the transition.

When the cine-image data stored in the cine-memory 110 before the transition is not retained (step S33; NO), the controller 108 accepts a touch input on the button 550 or 560 as a dual U/D button from the examiner through the touch panel 107b, transitions the display screen 220 into the display screen 270 or 280 according to the touch input button and clears the cine-memory 110 to be divided into the memory regions 110b and 110c (step S34). The live image is displayed in the ultrasound image region 441 in the display screen 270 or the ultrasound image region 452 in the display screen 280. Herein, the examiner presses the ultrasound probe 1b against a part of the test subject, which is scanned first. For example, to ultimately set the upper side as the healthy part side and the lower side as the affected part side, the examiner presses the ultrasound probe 1b against a site of the healthy part side when the display screen 270 with the active ultrasound image region 441 on the upper side is being displayed, and presses the ultrasound probe 1b against a site of the affected part side when the display screen 280 with the active ultrasound image region 452 on the lower side is being displayed.

Then, the controller 108 generates ultrasound image data of the site of the healthy part side or the affected part side, against which the ultrasound probe 1b is pressed in the step S34, displays the data as a live image in the ultrasound image region 421 or 432 and stores the ultrasound image data as cine-image data in the memory region 110b or 110c of the cine-memory 110 (step S35).

Then, the controller 108 accepts a touch input on the button 590 or 600, which is a dual U/D button and not disabled, from the examiner through the touch panel 107b, transitions the display screen 270 or 280 into the display screen 320 or 310 according to the touch input button, starts to display the live ultrasound image data started to be newly scanned in the ultrasound image region 492 or 481 (opposite side of the live image display side until the step S35), stops storing the new cine-image data in, for example, the memory region 110b or 110c of the cine-memory 110 which has last been storing the cine-image data, and starts to store the cine-image data of the live image in the memory region 110b or 110c which has not last been storing the cine-image data (step S36).

In the step S36, while the display screen 310 is displayed, the last frame of the cine-image data stored in the memory region 110b or 110c in the step S35 is displayed as a freeze image in the ultrasound image region 482. Similarly, while the display screen 320 is displayed, the last frame of the cine-image data stored in the memory region 110b or 110c in the step S35 is displayed as a freeze image in the ultrasound image region 491. Moreover, the examiner presses the ultrasound probe 1b against a part of the test subject not scanned in the step S35. For example, the examiner presses the ultrasound probe 1b against a site of the healthy part side when the display screen 310 with the active ultrasound image region 461 on the upper side is being displayed, and presses the ultrasound probe 1b against a site of the affected part side when the display screen 320 with the active ultrasound image region 492 on the lower side is being displayed.

When the cine-image data stored in the cine-memory 110 before the transition is retained after the transition (step S33; YES), the examiner presses the ultrasound probe 1b in advance against a part of the test subject, which is scanned first. For example, the examiner presses the ultrasound probe 1b against a site of the healthy part side when the display screen 310 with the active ultrasound image region 481 on the upper side is displayed next, and presses the ultrasound probe 1b against a site of the affected part side when the display screen 320 with the active ultrasound image region 492 on the lower side is displayed next. Herein, the controller 108 generates ultrasound image data of the site of the healthy part side or the affected part side, against which the ultrasound probe 1b is pressed, continues to display the data as a live image in the single ultrasound image region 410 and stores the ultrasound image data as cine-image data in the memory region 110b or 110c of the cine-memory 110 (step S37).

Then, the controller 108 accepts a touch input on the button 590 or 600 as a dual U/D button from the examiner through the touch panel 107b, transitions the display screen 240 into the display screen 310 or 320 according to the touch input button, starts to display the live image in the ultrasound image region 481 or 492, and starts to store the cine-image data of the live image in the memory region 110b or 110c (the memory region not storing the cine-image data in the step S37) of the cine-memory 110 (step S38). In the step S38, while the display screen 310 is displayed, the last frame of the cine-image data stored in the memory region 110b or 110c in the step S37 is displayed as a freeze image in the ultrasound image region 482. Similarly, while the display screen 320 is displayed, the last frame of the cine-image data stored in the memory region 110b or 110c in the step S37 is displayed as a freeze image in the ultrasound image region 491. Moreover, the examiner presses the ultrasound probe 1b against a part of the test subject not scanned in the step S37. For example, the examiner presses the ultrasound probe 1b against a site of the healthy part side when the display screen 310 with the active ultrasound image region 481 on the upper side is being displayed, and presses the ultrasound probe 1b against a site of the affected part side when the display screen 320 with the active ultrasound image region 492 on the lower side is being displayed.

After the step S36 or S38, the controller 108 generates ultrasound image data of the site of the healthy part side or the affected part side, against which the ultrasound probe 1b is pressed in the step S36 or S38, displays the data as a live image in the ultrasound image region 481 or 492 and starts to store the ultrasound image data as cine-image data in the memory region 110b or 110c (the memory region not storing the cine-image data in the step S35 or S37) of the cine-memory 110 (step S39). Then, the controller 108 accepts a touch input on a freeze button (not illustrated) on the display screen from the examiner through the touch panel 107b (step S40).

Then, the controller 108 stops scanning the ultrasound image and storing the cine-image data according the touch input on the freeze button in the step S40, displays a freeze image of the last frame of the cine-image data of the live image in the ultrasound image region 481 in the display screen 310 or the ultrasound image region 492 in the display screen 320 (step S41) and ends the vertical two-image display process.

As described above, according to the embodiments of the present invention, the ultrasound diagnostic imaging apparatus 1 includes the cine-memory 110 which stores the cine-image data of the live ultrasound image, and the controller 108 which determines whether or not to retain the cine-image data, which is stored in the cine-memory 110 before a transition from a display screen having one ultrasound image into a display screen having two images, after the transition, indicates, according to the determination result, the arrangement of the active ultrasound image in a display screen transitioned next and a setting state of retention of the cine-image data in the cine-memory 110 before and after the transition, generates the display screens 210 to 240 including the buttons 530 to 600 for accepting an input of the transition into the display screen, and displays the display screens on the display unit 107a.

Thus, by performing an input on the buttons 530 to 600 by the examiner with visual observation, the active ultrasound image (ultrasound image region) in the display screen of a transition destination and the retention of the cine-image data before and after the transition can be easily designated upon the transition from the display screen having one ultrasound image into the display screen having two images. Moreover, it is possible to easily and quickly scan the ultrasound image and enhance the productivity of the ultrasound image.

Also, the controller 108 generates a display screen including the button 520 for accepting an input as to whether or not to retain the cine-image data, which is stored in the cine-memory 110 before the transition, after transition, displays the display screen on the display unit 107a, and determines, according to an input state of the button 520, a setting as to whether or not to retain the cine-image data, which is stored in the cine-memory 110 before the transition, after the transition. Therefore, it is possible to easily set and easily determine whether or not to retain the cine-image data, which is stored in the cine-memory 110 before the transition, after the transition.

Furthermore, the controller 108 sets the input on the button 520 to be disabled after the transition into the display screens 250 to 320 having two images. Therefore, it is possible to prevent unnecessary button inputs and further enhance operability.

Moreover, the controller 108 sets the input on the buttons 530 to 600 to be disabled for a transition into a display screen, where the same active image is arranged, after the transition into the display screens 250 to 320 having two images. Therefore, it is possible to prevent unnecessary button inputs and further enhance operability.

In addition, the controller 108 divides the memory region of the cine-memory 110 into two memory regions including a memory region storing the last cine-image data before the transition when the cine-image data stored in the cine-memory 110 before the transition is determined to be retained after the transition. Therefore, it is possible to securely retain the cine-image data in one memory region as well as store new cine-image in the other memory region without preventing the retention.

Moreover, the controller 108 sets buttons for the transition to the buttons 530 to 560, which accept a transition into the display screens 250 to 280 including the ultrasound image and the blank image and accept an input as to whether to arrange the ultrasound image on the left or right side or on the upper or lower side when the cine-image data stored in the cine-memory 110 before the transition is determined not to be retained after the transition. The controller 108 sets buttons for the transition to the buttons 570 to 600, which accept a transition into the display screens 290 to 320 including two ultrasound images, the active ultrasound image and the inactive ultrasound image, and accept an input as to whether to arrange the active ultrasound image on the left or right side or on the upper or lower side when the cine-image data stored in the cine-memory 110 before the transition is determined to be retained after the transition. Therefore, to generate and display two ultrasound images, a healthy part side and an affected part side, the examiner can freely select an operation method which uses the display screens 250 to 280 and emphasizes the visibility of the ultrasound image by the blank image or an operation method which uses the display screens 290 to 320 and emphasizes a less number of operations.

Furthermore, the controller 108 make the display screens 210 to 240 including the buttons 530 to 600 include the button 510 which accepts an input as to switch the display between the buttons 530, 540, 570 and 580 corresponding to the transition into the display screen in which two images are arranged horizontally and the buttons 550, 560, 590 and 600 corresponding to the transition into the display screen in which two images are arranged vertically. Since the setting depth is shallow or deep depending on an observation subject, two vertical images may be appropriate in some cases, and two horizontal images maybe appropriate in other cases. Therefore, upon the transition into the display screen having two images, the horizontal or vertical arrangement of the ultrasound image can be freely designated.

Note that the descriptions in the above embodiments are examples of a preferred ultrasound diagnostic imaging apparatus according to the present invention and the present invention is not limited thereto.

For example, in the above embodiments, the configurations of displays of the buttons and the display screens upon the transitions from the display screens 210 to 240 having one ultrasound image into the display screens 250 to 320 having two images have been described, but are not limited thereto. For example, the configurations may be applied to displays of buttons and display screens upon a transition from a display screen having one ultrasound image into a display screen having three or more (e.g., four) images. In addition, the configurations may be applied to displays of buttons and display screens upon a transition from a display screen having a plurality of images including an ultrasound image into a display screen having more images than the plurality of the images. For example, the transition is a transition from a display screen having two images including an ultrasound image into a display screen having four images.

It is also possible to appropriately change the detailed configuration and the detailed operations of each unit constituting the ultrasound diagnostic imaging apparatus 1 in the above embodiments within a scope not departing from the gist of the present invention.

Although the present invention has been described and illustrated in detail, it is clearly understood that the same is by way of illustrated and example only and is not to be taken by way of limitation, the scope of the present invention being interpreted by terms of the appended claims.

What is claimed is:

1. An ultrasound diagnostic imaging apparatus which generates ultrasound image data based on a reception signal generated by an ultrasound probe, which transmits and receives ultrasound to and from a test subject, and displays an ultrasound image, the apparatus comprising:
a cine-memory that stores cine-image data of the ultrasound image which is live;
a determiner that determines whether or not to retain the cine-image data, which is stored in the cine-memory before a transition from a display screen having the ultrasound image into a display screen having a predetermined number of more images, after the transition; and
a hardware processor that indicates, according to a determination result of the determiner, an arrangement of the ultrasound image, which is active, in a display screen transitioned next and a setting state of retention of the cine-image data in the cine-memory before and after the transition, generates a display screen including a first button for accepting an input of the transition into the display screen, and displays the display screen on a display.

2. The ultrasound diagnostic imaging apparatus according to claim 1, wherein
the hardware processor generates a display screen including a second button for accepting an input as to whether or not to retain the cine-image data, which is stored in the cine-memory before the transition, after transition and displays the display screen on the display, and
the determiner determines, according to an input state of the second button, a setting as to whether or not to retain the cine-image data, which is stored in the cine-memory before the transition, after the transition.

3. The ultrasound diagnostic imaging apparatus according to claim 2, wherein
the hardware processor sets an input on the second button to be disabled after the transition into the display screen having the predetermined number of the images.

4. The ultrasound diagnostic imaging apparatus according to claim 1, wherein
the hardware processor sets an input on the first button to be disabled for a transition into a display screen, where a same active image is arranged, after the transition into the display screen having the predetermined number of the images.

5. The ultrasound diagnostic imaging apparatus according to claim 1, wherein
the hardware processor divides the cine-memory into a predetermined number of memory regions including a memory region storing last cine-image data before the transition when the cine-image data stored in the cine-memory before the transition is determined to be retained after the transition by the determiner.

6. The ultrasound diagnostic imaging apparatus according to claim 1, wherein
the predetermined number is two.

7. The ultrasound diagnostic imaging apparatus according to claim 6, wherein
the hardware processor sets the first button as a button which accepts a transition into a display screen including the ultrasound image and a blank image and accepts an input as to whether to arrange the ultrasound image on a left or right side or on an upper or lower side when the cine-image data stored in the cine-memory before the transition is determined not to be retained after the transition by the determiner, and sets the first button as a button which accepts a transition into a display screen including two ultrasound images, an active ultrasound image and an inactive ultrasound image, and accepts an input as to whether to arrange the active ultrasound image on the left or right side or on the upper or lower side when the cine-image data stored in the cine-memory before the transition is determined to be retained after the transition by the determiner.

8. The ultrasound diagnostic imaging apparatus according to claim 6, wherein
the hardware processor makes the display screen including the first button include a third button which accepts an input as to switch a display between the first button corresponding to the transition into the display screen in which two images are arranged horizontally and the first button corresponding to the transition into the display screen in which the two images are arranged vertically.
